(19) United States
(12) Patent Application Publication
(10) Pub. No.: US 2017/0335217 A1
(43) Pub. Date: Nov. 23, 2017

(54) ALKYLATION OF METALLOCENE-OLIGOMER WITH ISOALKANE TO MAKE HEAVY BASE OIL

(71) Applicant: Chevron U.S.A. Inc., San Ramon, CA (US)

(72) Inventor: Sven Ivar Hommeltoft, Pleasant Hill, CA (US)

(21) Appl. No.: 15/158,916

(22) Filed: May 19, 2016

(51) Int. Cl. C10M 105/04; C07C 2/32; C07C 2/62

(52) U.S. Cl. CPC C10M 105/04; C07C 2/62; C07C 2/32; C07C 2531/22; C10M 2203/022; C07C 2531/02

(57) ABSTRACT

A process to make an isoalkane alkylate base oil, comprising:
a. oligomerizing an olefin feed having a carbon number from 3 to 6 using a metallocene catalyst to make an unsaturated olefin oligomer; and
b. alkylating an isoalkane feed with the unsaturated olefin oligomer in the presence of an acidic alkylation catalyst, and without any addition of hydrogen, to make an alkylate product comprising the isoalkane alkylate base oil having a kinematic viscosity at 100° C. greater than 10 mm²/s, a VI higher than 80, and a bromine index less than 1000 mg Br/100 g.

This application is related to a co-filed application titled “HIGH VISCOSITY INDEX LUBRICANTS BY ISOALKANE ALKYLATION”, herein incorporated in its entirety.

### TECHNICAL FIELD

This application is directed to a process for oligomerizing olefins with a metallocene catalyst followed by alkylation of the metallocene-oligomer to make an isoalkane alkylate base oil.

### SUMMARY

This application provides a process to make an isoalkane alkylate base oil, comprising:
a. oligomerizing an olefin feed having a carbon number from 3 to 6 using a metallocene catalyst to make an unsaturated olefin oligomer; and
b. alkylating an isoalkane feed with the unsaturated olefin oligomer in the presence of an acidic alkylation catalyst, and without any addition of hydrogen, to make an alkylate product comprising the isoalkane alkylate base oil having a kinematic viscosity at 100° C. greater than 10 mm²/s, a VI higher than 80, and a bromine index less than 1000 mg Br/100 g.

The present invention may suitably comprise, consist of, or consist essentially of, the elements in the claims, as described herein.

### GLOSSARY

“Oligomer” refers to a compound that is intermediate between a monomer and a polymer, normally having a specified number of monomer units between four and a hundred.

“Base oil” refers to a hydrocarbon fluid to which other oils or substances are added to produce a lubricant.

“Lubricant” refers to substances (usually a fluid under operating conditions) introduced between two moving surfaces so as to reduce the friction and wear between them.

“Heavy base oil” in the context of this disclosure refers to a base oil having a kinematic viscosity at 100° C. greater than 10 mm²/s.

“Bright stock” refers to a heavy base oil having a kinematic viscosity above 180 mm²/s at 40° C., such as above 250 mm²/s at 40° C., or possibly ranging from 400 to 5000 mm²/s at 40° C.

“Cut point” refers to the temperature on a True Boiling Point (TBP) curve at which a predetermined degree of separation is reached.

“TBP” refers to the boiling point of a hydrocarbonaceous feed or product, as determined by Simulated Distillation (SIMDIST).

“Hydrocarbonaceous” means a compound or substance that contains hydrogen and carbon atoms, and which can include heteroatoms such as oxygen, sulfur, or nitrogen.

“Middle distillates” include products having cut points from 300° F. (149° C.) to 700° F. (371° C.). Middle distillates can include jet, kerosene, and diesel. Some typical naphthas and middle distillates for the North American market include the following:

| | Typical Cut Points, ° F. (° C.) |
|---|---|
| Products | for North American Market |
| Light Naphtha | C₅-180 (C₅-82) |
| Heavy Naphtha | 180-300 (82-149) |
| Jet | 300-380 (149-193) |
| Kerosene | 380-530 (193-277) |
| Diesel | 530-700 (277-371) |

An “isoalkane” is a hydrocarbon with the general formula CnH2n+2, n≧4 characterized by having at least one branch point, which means that the molecule contain at least one carbon atom bonded to three other carbon atoms and one hydrogen atom. The general formula for an isoalkane may be written as CHRR′R″, wherein R, R′ and R″ are linear or branched alkyl groups.

“Linear olefins” are unsaturated molecules with a linear hydrocarbon structure, and without any molecular branches.

“Alpha olefin” refers to any olefin having at least one terminal unconjungated carbon-carbon double bond.

“Viscosity index” (VI) represents the temperature dependency of a lubricant, as determined by ASTM D2270-10(E2011).

“Predominantly” refers to greater than 50 wt % in the context of this disclosure.

“Essentially” refers to from 90 wt % to 100 wt % in the context of this disclosure.

“API Base Oil Categories” are classifications of base oils that meet the different criteria shown in Table 1:

TABLE 1

| API Group | Sulfur, wt % | Saturates, wt % | Viscosity Index |
|---|---|---|---|
| I | >0.03 and/or | <90 | 80-119 |
| II | ≦0.03 and | ≧90 | 80-119 |
| III | ≦0.03 and | ≧90 | ≧120 |

| | |
|---|---|
| IV | All Polyalphaolefins (PAOs) |
| V | All base oils not included in Groups I-IV(naphthenics, |
| | non-PAO synthetics) |

“Group II+” is an unofficial, industry-established ‘category’ that is a subset of API Group II base oils that have a VI greater than 110, usually 112 to 119.

“Group III+” is another unofficial, industry-established ‘category’ that is a subset of API Group III base oils that have a VI greater than 130.

“Catalytic dewaxing”, or “hydroisomerization dewaxing”, refers to a process in which normal paraffins are isomerized to their more branched counterparts in the presence of hydrogen and over a catalyst.

“Kinematic viscosity” refers to the ratio of the dynamic viscosity to the density of an oil at the same temperature and pressure, as determined by ASTM D445-15.

“LHSV” means liquid hourly space velocity.

“Periodic Table” refers to the version of the IUPAC Periodic Table of the Elements dated Jun. 22, 2007, and the numbering scheme for the Periodic Table Groups is as described in Chemical And Engineering News, 63(5), 27 (1985).

“Bromine index” refers to the amount of bromine-reactive material in petroleum hydrocarbons and is a measure of trace amounts of unsaturates in these materials. Bromine index is reported in mg Br/100 g of sample.

“Metallocene catalyst” refers to a positively charged metal ion sandwiched between two negatively charged cyclopentadienyl anions that can catalyze a polymerization or an oligomerization reaction.

“Acidic ionic liquid” refers to materials consisting entirely of ions, that can donate a proton or accept an electron pair in reactions, and that are liquid below 100° C.

### DETAILED DESCRIPTION

Metallocene Catalyst

Known catalysts can be used as the metallocene catalyst employed in the oligomerizing. For example, the metallocene catalyst can comprise a combination of (a) a metallocene complex containing a transition metal selected from the group consisting of titanium (Ti), zirconium (Zr), hafnium, (Hf), and combinations thereof: (b) a compound or a combination of compounds that are an activator that activate the metallocene complex to make it an active catalyst; and (c) an optional co-activator that serves as alkyl group donor. Chemically the activation of the metallocene complex may involve abstraction of a weakly basic ligand, such as a halide ligand, for instance a chloride ligand from the metallocene complex and optionally replace it with an alkyl ligand, or a hydride ligand that by insertion of an olefin molecule may be converted to an alkyl ligand.

In one embodiment, the metallocene catalyst comprises a metallocene complex containing a transition metal selected from the group of titanium (Ti), zirconium (Zr), hafnium, (Hf), and combinations thereof.

In one embodiment the metallocene complex comprises a conjugated 5-membered carbon ring, such as metallocene complexes having a substituted or an unsubstituted cyclopentadienyl ligand.

Examples of the metallocene complex serving as the catalyst component (a) include known compounds, specifically, bis(n-octadecylcyclopentadienyl)zirconium dichloride, bis(trimethylsilylcyclopentadienyl)zirconium dichloride, bis(tetrahydroindenyl)zirconium dichloride, bis[(t-butyldimethylsilyl)cyclopentadienyl]zirconium dichloride, bis(di-t-butylcyclopentadienyl)zirconium dichloride, ethylidenebis(indenyl)zirconium dichloride, biscyclopentadienylzirconium dichloride, ethylidenebis(tetrahydroindenyl)zirconium dichloride, and bis[3,3-(2-methyl-benzindenyl)]dimethylsilanediylzirconium dichloride, (1,2′-dimethylsilylene)(2,1′-dimethylsilylene)bis(3-trimethylsilylmethylindenyl)zirconium dichloride, bis(1,3-dimethylcyclopentadienyl) zirconium dichloride, bis(t-butylcyclopentadienyl)zirconium dichloride [(t-BuCp)₂ZrCl₂], bis(i-propylcyclopentadienyl)zirconium dichloride , bis(n-butylcyclopentadienyl)zirconium dichloride, bis(ethyl cyclopentadienyl)zirconium dichloride, bis(pentamethylcyclopentadienyl)zirconium dichloride and dimethysilyl bis[cyclopentadienyl]zirconium dichloride .

These metallocene complexes may be used singly or in combination with each other.

In one embodiment, the activator (b) can be a Lewis acid that serves to abstract the weakly basic ligand from the metallocene and the co-activator (c) can be an alkyl group containing metal-organic compound, such as a trialkylaluminum compound, capable of donating the alkyl group to the metallocene complex once the weakly basic ligand is removed.

One type of activator (b-1) that can be used in metallocene catalysis include methyl aluminoxane (MAO), modified methyl aluminoxane (MMAO), ethylaluminoxane, butylaluminoxane, isobutylaluminoxane, and cyclic aluminoxanes. These aluminoxanes can be used singly or as mixtures thereof.

In one embodiment,the aluminoxane activators can be used in large excess relative to the metallocene complex. MAO and MMAO can play a dual role as both activator for the metallocene complex and as an alkyl group donor and thus can be used in some embodiments without an additional co-activator.

Another type of activator (b-2) that can be used in some embodiments is known as a stoichiometric activator. Stoichiometric activators are named as such since they in principle react in a well-defined stoichiometric reaction with the metallocene complex containing the weakly basic ligand, as opposed to the MAO and MMAO type activators. Examples of stoichiometric activators are strong Lewis acids such as for instance perfluorinated trialkylboron or perfluorinated triarylboron. Also ionic stoichiometric activators comprising hydrocarbon soluble salts of non-coordinating anions, such as for instance tetraphenylborate or tetrakis(pentafluorophenyl)borate or tetrakis(perfluoronaphthyl)borate, with an acidic bulky cations such as for instance trialkylammonium, dialkylammonium or N,N dialkylanilinium may be used as activators The stoichiometric activators can be used singly or in combination with each other.

Yet another group of activators (b-3) that can be used in some embodiments are solid super acids, which may for instance be fluorinated silica-alumina. Both stoichiometric activators, ionic as well as non-ionic, and solid super acid activators can be used with an alkyl group donor (such as a trialkylaluminum).

In one embodiment, as the catalyst component (b), one or more compounds (b-1) or one or more compounds (b-2) or one or more compounds (b-3) may be used. Alternatively, one or more compounds (b-1) and one or more compounds (b-2) may be used in combination.

In one embodiment, when the compound (b-1) is employed as the catalyst component (b), the ratio by mole of catalyst component (a) to catalyst component (b) can be 1:1 to 1:100, such as 1:1 to 1:10. In one embodiment, when the compound (b-2) is employed, the mole ratio can be 1:1 to 1:1,000,000, such as 1:10 to 1:10,000. In one embodiment, when the compound (b-3) is employed, the mole ratio can be 1:1 to 1:1,000,000, such as 1:10 to 1:10,000.

Examples of the organic aluminum compound serving as the optional metallocene catalyst component (c) include trimethylaluminum, triethylaluminum, triisopropylaluminum, triisobutylaluminum, dimethylaluminum chloride, diethylaluminum chloride, methylaluminum dichloride, ethylaluminum dichloride, dimethylaluminum fluoride, diisobutylaluminum hydride, diethylaluminum hydride, ethylaluminum sesquichloride, and combinations thereof.

In one embodiment, when the metallocene catalyst components (a) and (c) are employed, the ratio by mole of catalyst component (a) to catalyst component (c) can be 1:1 to 1:10,000, such as 1:5 to 1:2,000, or such as 1:10 to 1:1,000. Through employment of the metallocene catalyst component (c), oligomerization activity per amount of transition metal can be enhanced to produce an oligomer with a higher carbon number.

In one embodiment when metallocene components of type (a) are employed together with catalyst component (b-1) (MAO or MMAO) the (b-1) component or some of the (b-1) catalyst component is added to the feed stream prior to introducing the metallocene complex. In this embodiment part of the excess of (b-1) can serve as a scavenger for impurities in the olefin feed.

In one embodiment, when metallocene components (a), (b-2 or b-3) and co-activator catalyst component (c) are used, part or all of catalyst component (c) can be added to the olefin feed to scavenge impurities before introducing the olefin feed to the metallocene component (a).

In one embodiment, when the metallocene catalyst comprises components (a) and (b), oligomerizing can be performed in an inert gas atmosphere such as nitrogen.

In one embodiment, the oligomerizing is performed in the presence of hydrogen at a hydrogen pressure from 200 to 5000 kPa.

The metallocene catalyst components may be prepared in a metallocene catalyst preparation tank before use, or during the oligomerizing.

The olefin feed has a carbon number from 3 to 6. In one embodiment, the olefin feed comprises a propylene, a butene, or a mixture thereof. The olefin feed can come from a number of sources, including: as a byproduct from the steam cracking of liquid feedstocks, such as propane, butane, gas condensates, naphtha and LPG; from LPG range products produced in a FCC unit in a refinery; from propane or butane; and by metathesis.

The oligomerizing can be performed in a batch manner or a continuous manner. In one embodiment, the oligomerizing requires no particular solvent and may be performed in suspension, in the olefin feed, or in an inert solvent. In one embodiment, the olefin feed comprises an inert hydrocarbon solvent. Examples of inert hydrocarbon solvents are C3-C6 alkanes, such as propane. In one embodiment, the oligomerizing is performed with a liquid aromatic solvent such as benzene, ethylbenzene, or toluene. In one embodiment the oligomerizing is performed in a reaction mixture where the olefin feed is present in an excessive amount.

Oligomerizing can be performed at about 10° C. to about 150° C., under atmospheric pressure to about 20000 kPa. In one embodiment, the metallocene catalyst can be used in an amount with respect to the olefin feed; i.e., a mole ratio of olefin feed/metallocene complex of 2,000/l to 2,000,000/l. In one embodiment, the oligomerizing reaction time can be from 5 minutes to 50 hours.

In one embodiment, the oligomerizing can be followed by a post-treatment prior to the alkylating. In the post-treatment, the oligomerizing reaction system can be deactivated through a known method, for example, by adding water or alcohol thereto, to thereby terminate the oligomerizing. In one embodiment the oligomerizing reaction system can be de-ashed by use of an aqueous alkaline solution or an alcoholic alkaline solution.

In one embodiment, between the oligomerizing and alkylating, the unsaturated olefin oligomer can be washed for neutralization, filtered, centrifuged, or distilled. Unreacted hydrocarbons and undesired olefin isomers that can be by-produced during the oligomerizing can be removed through stripping, whereby the unsaturated olefin oligomer is obtained.

In one embodiment, the unsaturated olefin oligomer produced in the presence of a metallocene catalyst comprises a double bond. For example, the unsaturated olefin oligomer can have a bromine number from 5-15. Bromine number can be measured by ASTM D1159-07(R 2012). In one embodiment, the unsaturated olefin oligomer comprises a terminal vinylidene bond.

In one embodiment, the unsaturated olefin oligomer has a kinematic viscosity at 100° C. from 10 to 80 mm²/s.

Isoalkane Feed

In one embodiment, the isoalkane feed comprises a mixture of isoalkanes.

In one embodiment, the isoalkane feed comprises isoalkanes have nine or less carbons. In one embodiment, the isoalkane feed comprises isobutane, isopentane, hydrocracker naphtha, or mixtures thereof.

Alkylating

The process to make an isoalkane alkylate base oil comprises alkylating the isoalkane feed with the olefin feed in the presence of an acidic alkylation catalyst under alkylation conditions. The alkylating can be done at an alkylation temperature greater than −20° C., such as from −15° C. to 100° C., or from −10° C. to 50° C.

Oxidative stability, and light or UV stability of base oils can improve when the amount of unsaturation, double bonds, or olefinic content, is reduced by the alkylating. In one embodiment, it is not necessary to further hydrotreat the isoalkane alkylate base oil as it has a high degree of saturation.

In one embodiment, the acidic alkylation catalyst is selected from the group consisting of an acidic ionic liquid, a sulfuric acid, a hydrofluoric acid, a triflic acid, another Brønsted acid with a Hammet acidity function less than −10 (H₀<−10), an acidic zeolite, a sulfated zirconia, and a tungstated zirconia. The Hammett acidity function (H₀) is a measure of acidity that is used for very concentrated solutions of strong acids, including superacids. It was proposed by the physical organic chemist Louis Plack Hammett and is the best-known acidity function used to extend the measure of Brønsted-Lowry acidity beyond the dilute aqueous solutions for which the pH scale is useful.

Zeolites useful for alkylating isoalkanes include large pore zeolites such as for instance zeolite X and zeolite Y and zeolite beta, in their proton form or rare earth exchanged form.

In one embodiment, the acidic alkylation catalyst comprises an ionic liquid catalyst and a Brønsted acid. In this embodiment, the Brønsted acid acts as a promoter or co-catalyst. Examples of Brønsted acids are sulfuric acid, HCl, HBr, HF, phosphoric acid, HI, etc. Other strong acids that are proton donors can also be suitable Brønsted acids. In one embodiment, the Brønsted acid is produced internally within the process by the conversion of an alkyl halide into the corresponding hydrogen halide. In one embodiment the Brønsted acid is formed by a reaction of a Lewis acid component of an ionic liquid, such as chloroaluminate ions for instance reacting with a weakly acidic proton donor such as an alcohol or water to form HCl.

Acidic Ionic Liquid

Examples of acidic ionic liquid catalysts and their use for alkylation of paraffins with olefins are taught, for example, in U.S. Pat. Nos. 7,432,408 and 7,432,409, 7,285,698, and U.S. patent application Ser. No. 12/184069, filed Jul. 31, 2008. In one embodiment, the acidic ionic liquid is a composite ionic liquid catalyst, wherein the cations come from a hydrohalide of an alkyl-containing amine or pyridine, and the anions are composite coordinate anions coming from two or more metal compounds.

The most common acidic ionic liquids are those prepared from organic-based cations and inorganic or organic anions. The acidic ionic liquid is composed of at least two components which form a complex. The acidic ionic liquid comprises a first component and a second component. The first component of the acidic ionic liquid will typically comprise a Lewis acid compound selected from components such as Lewis acid compounds of Group 13 metals, including aluminum halides, alkyl aluminum dihalides, gallium halide, and alkyl gallium halide (see the Periodic Table, which defines the elements that are Group 13 metals). Other Lewis acid compounds besides those of Group 13 metals may also be used. In one embodiment the first component is aluminum halide or alkyl aluminum dihalide. For example, aluminum trichloride (AlCl₃) may be used as the first component for preparing the ionic liquid catalyst. In one embodiment, the alkyl aluminum dihalides that can be used can have the general formula Al₂X₄R₂, where each X represents a halogen, selected for example from chlorine and bromine, each R represents a hydrocarbyl group comprising 1 to 12 atoms of carbon, aromatic or aliphatic, with a branched or a linear chain. Examples of alkyl aluminum dihalides include dichloromethylaluminum, dibromomethylaluminum, dichloroethylaluminum, dibromoethylaluminum, dichloro n-hexylaluminum, dichloroisobutylaluminum, either used separately or combined.

The second component making up the acidic ionic liquid can be an organic salt or mixture of salts. These salts may be characterized by the general formula Q+A−, wherein Q+ is an ammonium, phosphonium, boronium, oxonium, iodonium, or sulfonium cation and A− is a negatively charged ion such as Cl⁻, Br⁻, ClO₄⁻, NO₃⁻, BF₄⁻, BCl₄⁻, PF₆⁻, SbF₆⁻, AlCl₄⁻, Al₂Cl₇⁻, Al₃Cl₁₀⁻, GaCl₄⁻, Ga₂Cl₇⁻, Ga₃Cl₁₀⁻, AsF₆⁻, TaF6⁻, CuCl₂⁻, FeCl₃⁻, AlBr₄⁻, Al₂Br₇⁻, Al₃Br₁₀⁻, SO₃CF₃⁻, and 3-sulfurtrioxyphenyl.

In one embodiment the second component is selected from those having quaternary ammonium halides containing one or more alkyl moieties having from about 1 to about 9 carbon atoms, such as, for example, trimethylammonium hydrochloride, methyltributylammonium, 1-butyl pyridinium, or alkyl substituted imidazolium halides, such as for example, 1-ethyl-3-methyl-imidazolium chloride.

In one embodiment, the acidic ionic liquid comprises a monovalent cation selected from the group consisting of a pyridinium ion, an imidazolium ion, a pyridazinium ion, a pyrazolium ion, an imidazolinium ion, a imidazolidinium ion, an ammonium ion, a phosphonium ion, and mixtures thereof. Examples of possible cations (Q+) include a butylethylimidazolium cation [beim], a butylmethylimidazolium cation [bmim], butyldimethylimidazolium cation [bmmim], decaethylimidazolium cation [dceim], a decamethylimidazolium cation [dcmim], a diethylimidazolium cation [eeim], dimethylimidazolium cation [mmim], an ethyl-2,4-dimethylimidazolium cation [e-2,4-mmim], an ethyldimethylimidazolium cation [emmim], an ethylimidazolium cation [eim], an ethylmethylimidazolium [emim] cation, an ethylpropylimidazolium cation [epim], an ethoxyethylmethylimidazolium cation [etO-emim], an ethoxydimethylimidazolium cation [etO-mmim], a hexadecylmethylimidazolium cation [hexadmim], a heptylmethylimidazolium cation [hpmim], a hexaethylimidazolium cation [hxeim], a hexamethylimidazolium cation [hxmim], a hexadimethylimidazolium cation [hxmmim], a methoxyethylmethylimidazolium cation [meO-emim], a methoxypropylmethylimidazolium cation [meO-prmim], a methylimidazolium cation [mim], dimethylimidazolium cation [mmim], a methylnonylimidazolium cation [mnim], a methylpropylimidazolium cation [mpim], an octadecylmethylimidazolium cation [octadmim], a hydroxylethylmethylimidazolium cation [OH-emim], a hydroxyloctylmethylimidazolium cation [OH-omim], a hydroxylpropylmethylimidazolium cation [OH-prmim], an octylmethylimidazolium cation [omim], an octyldimethylimidazolium cation [ommim], a phenylethylmethylimidazolium cation [ph-emim], a phenylmethylimidazolium cation [ph-mim], a phenyldimethylimidazolium cation [ph-mmim], a pentylmethylimidazolium cation [pnmim], a propylmethylimidazolium cation [prmim], a 1-butyl-2-methylpyridinium cation[1-b-2-mpy], 1-butyl-3-methylpyridinium cation[1-b-3-mpy], a butylmethylpyridinium [bmpy] cation, a 1-butyl-4-dimethylacetylpyridinium cation [1-b-4-DMApy], a 1-butyl-4−35 methylpyridinium cation[1-b-4-mpy], a 1-ethyl-2-methylpyridinium cation[1-e-2-mpy], a 1-ethyl-3-methylpyridinium cation[1-e-3-mpy], a 1-ethyl-4-dimethylacetylpyridinium cation[1-e-4-DMApy], a 1-ethyl-4-methylpyridinium cation[1-e-4-mpy], a 1-hexyl-5 4dimethylacetylpyridinium cation[1-hx-4-DMApy], a 1-hexyl-4-methylpyridinium cation[1-hx-4-mpy], a 1-octyl-3-methylpyridinium cation[1-o-3-mpy], a 1-octyl-4-methylpyridinium cation[1-o-4-mp y], a 1-propyl-3-methylpyridinium cation[1-pr-3-mpy], a 1-propyl-4-methylpyridinium cation[1-pr-4-mpy], a butylpyridinium cation [bpy], an ethylpyridinium cation [epy], a heptylpyridinium cation [hppy], a hexylpyridinium cation [hxpy], a hydroxypropylpyridinium cation [OH-prpy], an octylpyridinium cation [opy], a pentylpyridinium cation [pnpy], a propylpyridinium cation [prpy], a butylmethylpyrrolidinium cation [bmpyr], a butylpyrrolidinium cation [bpyr], a hexylmethylpyrrolidinium cation [hxmpyr], a hexylpyrrolidinium cation [hxpyr], an octylmethylpyrrolidinium cation [ompyr], an octylpyrrolidinium cation [opyr], a propylmethylpyrrolidinium cation [prmpyr], a butylammonium cation [b-N], a tributylammonium cation [bbb-N], a tetrabutylammonium cation [bbbb-N], a butylethyldimethylammonium cation [bemm-N], a butyltrimethylammonium cation [bmmm-N], a N,N,N-trimethylethanolammonium cation [choline], an ethylammonium cation [e-N], a diethylammonium cation [ee-N], a tetraethylammonium cation [eeee-N], a tetraheptylammonium cation [hphphphp-N], a tetrahexylammonium cation [hxhxhxhx-N], a methylammonium cation [m-N], a dimethylammonium cation [mm-N], a tetramethylammonium cation [mmmm-N], an ammonium cation [N], a butyldimethylethanolammonium cation [OHe-bmm-N], a dimethylethanolammonium cation [OHe-mm-N], an ethanolammonium cation [OHe—N], an ethyldimethylethanolammonium cation [OHe-emm-N], a tetrapentylammonium cation [pnpnpnpn-N], a tetrapropylammonium cation [prprprpr-N], a tetrabutylphosphonium cation [bbbb-P], a tributyloctylphosphonium cation [bbbo-P], or combinations thereof.

In one embodiment, the second component is selected from those having quaternary phosphonium halides containing one or more alkyl moieties having from 1 to 12 carbon atoms, such as, for example, trialkyphosphonium hydrochloride, tetraalkylphosphonium chlorides, and methyltrialkyphosphonium halide.

In one embodiment, the acidic ionic liquid comprises an unsubstituted or partly alkylated ammonium ion.

In one embodiment, the acidic ionic liquid is chloroaluminate or a bromoaluminate. In one embodiment the acidic ionic liquid is a quaternary ammonium chloroaluminate ionic liquid having the general formula RR′ R″ N H+ Al₂C1₇⁻, wherein R, R′, and R″ are alkyl groups containing 1 to 12 carbons. Examples of quaternary ammonium chloroaluminate ionic liquids are an N-alkyl-pyridinium chloroaluminate, an N-alkyl-alkylpyridinium chloroaluminate, a pyridinium hydrogen chloroaluminate, an alkyl pyridinium hydrogen chloroaluminate, a di alkyl-imidazolium chloroaluminate, a tetra-alkyl-ammonium chloroaluminate, a tri-alkyl-ammonium hydrogen chloroaluminate, or a mixture thereof.

The presence of the first component should give the acidic ionic liquid a Lewis or Franklin acidic character. Generally, the greater the mole ratio of the first component to the second component, the greater is the acidity of the acidic ionic liquid.

For example, a typical reaction mixture to prepare n-butyl pyridinium chloroaluminate ionic liquid is shown below:

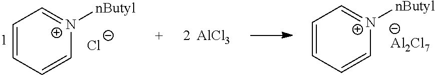

In one embodiment, the acidic ionic liquid utilizes a co-catalyst to provide enhanced or improved alkylation activity. Examples of co-catalysts include alkyl halide or hydrogen halide. A co-catalyst can comprise, for example, anhydrous HCl or organic chloride (see, e.g., U.S. Pat. Nos. 7,495,144 to Elomari, and U.S. Pat. No. 7,531,707 to Harris et al.). When organic chloride is used as the co-catalyst with the acidic ionic liquid, HCl may be formed in situ in the apparatus either during the alkylating or during post-processing of the output of the alkylating. In one embodiment, the alkylating with the acidic ionic liquid is conducted in the presence of a hydrogen halide, e.g., HCl.

The alkyl halides that may be used include alkyl bromides, alkyl chlorides and alkyl iodides. Such alkyl halides include but are not limited to isopentyl halides, isobutyl halides, t-butyl halides, n-butyl halides, propyl halides, and ethyl halides. Alkyl chloride versions of these alkyl halides can be preferable when chloroaluminate ionic liquids are used. Other alkyl chlorides or alkyl halides having from 1 to 8 carbon atoms can be also used. The alkyl halides may be used alone or in combination.

When used, the alkyl halide or hydrogen halide co-catalysts are used in catalytic amounts. In one embodiment, the amounts of the alkyl halides or hydrogen halide should be kept at low concentrations and not exceed the molar concentration of the AlCl₃ in the acidic ionic liquid. For example, the amounts of the alkyl halides or hydrogen halide used may range from 0.05 mol %-100 mol % of the Lewis acid AlCl₃ in the acidic ionic liquid in order to keep the acidity of the acidic ionic liquid catalyst at the desired performing capacity.

In one embodiment, the acidic alkylation catalyst comprises an ionic liquid catalyst and a Brønsted acid. In this embodiment, the Brønsted acid acts as a promoter or co-catalyst. Examples of Brønsted acids are sulfuric acid, HCl, HBr, HF, phosphoric acid, HI, etc. Other strong acids that are proton donors can also be suitable Brønsted acids. In one embodiment, the Brønsted acid is produced internally within the process by the conversion of an alkyl halide into the corresponding hydrogen halide.

In one embodiment, the process can additionally comprise recycling an excess of the isoalkane feed to the alkylating. For example, the process can include distilling out an excess isoalkane after the alkylating and then recycling the excess isoalkane to the alkylating.

In one embodiment, the process can additionally comprise neutralizing a residual acidic alkylation catalyst in the isoalkane alkylate base oil.

In one embodiment, the process can additionally comprise distilling the alkylate product and collecting the isoalkane alkylate base oil and a middle distillate. In one embodiment, the isoalkane alkylate product is the predominant cut that is collected by the distilling. The middle distillate can have a cetane number that makes it valuable as a diesel fuel. In one sub-embodiment, the middle distillate can have a DCN from 30 to 50, or a DCN from 33 to 45.

Isoalkane Alkylate Base Oil

The isoalkane alkylate base oil has a VI higher than 80, a kinematic viscosity at 100° C. greater than 10 mm²/s, and a bromine index less than 1000 mg Br/100g.

In one embodiment, the isoalkane alkylate base oil has a kinematic viscosity at 100° C. from greater than 10 mm²/s to 5000 mm²/s.

In one embodiment, the isoalkane alkylate base oil has a kinematic viscosity at 100° C. from greater than 10 mm²/s to 100 mm²/s.

In one embodiment, the isoalkane alkylate base oil comprises from zero to less than 5 wt % linear alkanes.

In one embodiment, the isoalkane alkylate base oil comprises from zero to less than 100 wppm aromatics. In a sub-embodiment, the isoalkane alkylate base oil can comprise from zero to less than 10 ppb aromatics.

In one embodiment, the isoalkane alkylate base oil comprises less from zero to less than 25 wppm sulfur. In a sub-embodiment, the isoalkane alkylate base oil can comprise from zero to less than 10 ppb sulfur. In one embodiment, the VI is greater than 90. In one embodiment the VI is from 91 to 120. In different embodiments, the isoalkane alkylate base oil is an API Group II or an API Group II+ base oil. In one embodiment, the isoalkane alkylate base oil is an API Group II bright stock.

In one embodiment, the isoalkane alkylate base oil comprises a mixture of isoalkanes, and in a sub-embodiment the isoalkane alkylate base oil can consist predominantly of a mixture of isoalkanes in the base oil boiling range.

The base oil boiling range is defined herein as a boiling point range between 550° F. (287.8° C.) and 1400° F. (760° C.), where in the lower value is the T5 boiling point and the upper value is the T95 boiling point. Boiling range can be measured by simulated distillation according to ASTM D2887-15 (E 2015), ASTM D 6352-15, or equivalent test methods that are appropriate for the boiling range of the sample being tested. An equivalent test method refers to any analytical method which gives substantially the same results as the standard method. T5 relates to the temperature at which 5 weight percent of the isoalkane base oil has a lower boiling point. T95 refers to the temperature at which 95 weight percent of the isoalkane alkylate base oil has a lower boiling point. The isoalkanes in the base oil boiling range are generally hydrocarbons with 18 to 100 carbon atoms.

In one embodiment, the isoalkane alkylate base oil comprises localized branching introduced partially from the isoalkane feed and partially from the alkylating.

The pour point, and other cold flow properties, of the isoalkane alkylate base oil can be excellent. For example, the pour point can be less than or equal to 0° C., less than −10° C., less than −15° C., less than −24° C., or even less than −50° C. Pour point can be determined by ASTM D5950-14, or by an equivalent test method. In one embodiment, the isoalkane alkylate base oil has a cloud point less than −10° C., such as less than −25° C., or even less than −60° C. Cloud point can be measured by ASTM D2500-16, or by an equivalent test method.

Bromine Index

In one embodiment, the isoalkane alkylate base oil has a bromine index from less than 100 to less than 1000 mg Br/100 g. In one embodiment, the isoalkane alkylate base oil has a bromine index less than 500 mg Br/100 g. The bromine index less than 1000 mg Br/100 g, or even less than 200 mg Br/100 g can be obtained prior to any subsequent hydrogenation.

Bromine index can be determined by proton Nuclear Magnetic Resonance (NMR). Proton NMR is generally taught in https:/en.wikipedia.org/wiki/Proton_nuclear_magnetic_resonance.

The following assumptions are made for the Bromine index determinations in test samples of alkylate base oil:
1) Residual olefins in the test sample are represented by the formula: R1R2C═CHR3, so that one vinylic hydrogen represents an olefin group.
2) The average carbon in the test sample caries two protons and thus may be represented by an average molecular wt of 14.0268 g/mole
3) All proton resonances in the range 0.5-0.95 represent methyl groups (3 protons per carbon)
4) All proton resonances in the range 0.95-1.40 ppm represent CH₂ groups (2 protons per carbon)
5) All proton resonances in the range 1.4-2.1 ppm represent CH groups (1 proton per carbon)
6) All proton resonances in the range 4-6 ppm represent RR′C═CHR″ groups (0.5 proton per carbon or one per double bond).
7) One double bond reacts with one equivalent of bromine, i.e., one mole of olefin reacts with one mole of dibromine (Br₂, MW=159.8 g/mole)

Integrals in the acquired proton NMR spectrum are represented by I(“group”) , e.g., the integral of a methyl group is I(CH3) and the integral of an olefin group is I(RR′C═CHR″).

Bromine number is defined as the amount of bromine (in g Br₂) needed to titrate all the olefins in 100 g of the test sample. Bromine index=1000*bromine number.

The bromine index is calculated from the proton NMR integrals with the following formula: Bromine index=1000*100*(159.8/14.0268)*I(RR′C+CHR″)/{0.3333*I(CH3)+0.5*I(CH2)+I(CH)+2*I(RR′C═CHR″)}.

The absence of any proton resonances in the NMR spectrum is interpreted as a bromine index <100, based on the sensitivity of the proton NMR spectrometer that is used.

In one embodiment, the isoalkane alkylate base oil has a bromine index less than 100.

Finished Lubricant

In one embodiment, the process additionally comprises blending the isoalkane alkylate base oil with at least one additive to make a finished lubricant. A wide variety of high quality finished lubricants can be made by blending the isoalkane alkylate base oil with at least one additive selected from the group consisting of antioxidants, detergents, anti-wear agents, metal deactivators, corrosion inhibitors, rust inhibitors, friction modifiers, anti-foaming agents, viscosity index improvers, demulsifying agents, emulsifying agents, tackifiers, complexing agents, extreme pressure additives, pour point depressants, and combinations thereof; wherein selection of the at least one additive is directed largely by the end-use of the finished lubricant being made, wherein said finished lubricant can be of a type selected from the group consisting of engine oils, greases, heavy duty motor oils, passenger car motor oils, transmission and torque fluids, natural gas engine oils, marine lubricants, railroad lubricants, aviation lubricants, food processing lubricants, paper and forest products, metalworking fluids, gear lubricants, compressor lubricants, turbine oils, hydraulic oils, heat transfer oils, barrier fluids, and other industrial products. In one embodiment, the alkylate base oil can be blended with at least one additive to make a multi-grade engine oil.

### EXAMPLES

### Example 1

### Metallocene Catalyzed Propylene Oligomerization

Metallocene catalyzed oligomerizations of hydrocarbon feeds were performed in an autoclave. All of the hydrocarbon feeds used in the oligomerizations were purified by passing them through 13× molecular sieve and Selexcorb CD alumina before use.

408.3 g. (0.9 lb.) of a mixture of 70 wt % propylene and 30 wt % propane was loaded into a 1 liter stirred batch autoclave and pressured with an additional 1379 kPa (200 psig) of hydrogen. 2 ml of a 0.0061 mmole/ml solution of (t-BuCp)₂ZrCl₂ in dry de-aerated toluene and 4 ml of a 1.151 mmole/ml solution of methylaluminoxane (MAO) in toluene was added to the autoclave through an injection port. The resulting reaction mixture was reacted with agitation at 70° C. for 2-3 hrs. After the end of the reaction, the autoclave was depressurized and the crude oligomer product was washed with water and centrifuged clear to yield about 300 g of an oligomer product with a bromine number of about 10 and a kinematic viscosity at 100° C. around 40-45 mm²/s. The high bromine number indicated that the metallocene—catalyzed oligomer product contained significant unsaturation, i.e., olefins. The bromine number of 10 was consistent with an average chain length of 110 carbons in the oligomers or an average molecular weight around 1500 g/mole. The metallocene-catalyzed oligomer product was predominantly terminal olefins based on their proton NMR

### Example 2

### Alkylation of Isopentane With Metallocene-Catalyzed Propylene Oligomer

30 ml n-butylpyridinium heptachlorodialuminate ionic liquid was loaded into a 1 liter mechanically agitated flask with 100 ml isopentane and 0.5 ml t-butyl chloride under an atmosphere of nitrogen. The stirred mixture was cooled on an ice bath to 3° C. A solution of 150 ml of the propylene oligomer product from Example 1 and 1 ml t-butyl chloride in 500 ml isopentane was added to the flask over a period of 25 minutes, which maintaining a reaction temperature of 3-6° C. The mixture was stirred for another 5 minutes, at which point the agitation was stopped allowing the ionic liquid layer to settle out.

The hydrocarbon phase product was siphoned out of the flask. An additional batch of 100 ml propylene oligomer product from Example 1, 1 ml t-butyl chloride, and 400 ml isopentane were added as described earlier to make a second batch of hydrocarbon products. These steps were repeated to make a total of 5 product batches. Each hydrocarbon phase product was washed with dilute aqueous sodium bicarbonate to neutralize and remove residual HCl and chloroaluminate.

The washed hydrocarbon phase products were combined and unreacted isopentane was removed by RotoVap. The washed combined hydrocarbon phase products were an isoalkane alkylate product. The isoalkane alkylate product was distilled to yield 19.9 g naphtha (Bp<50° C. at 80 torr), 111.7 g middle distillate, and 295.7 g isoalkane alkylate base oil fractions. The SIMDIST data for the middle distillate and base oil fractions are shown in Table 1.

TABLE 1

| | Middle Distillate | Isoalkane Alkylate Base |
|---|---|---|
| Wt % | Fraction | Oil Fraction |
| 0.1 | 309° F. (153.9° C.) | 497° F. (258.3° C.) |
| 0.5 (IBP) | 311° F. (155° C.) | 513° F. (267.2° C.) |
| 5 | 316° F. (157.8° C.) | 597° F. (313.9° C.) |
| 10 | 321° F. (160.6° C.) | 670° F. (354.4° C.) |
| 15 | 334° F. (167.8° C.) | 742° F. (394.4° C.) |
| 20 | 338° F. (170° C.) | 855° F. (457.2° C.) |
| 25 | 342° F. (172.2° C.) | 994° F. (534.4° C.) |
| 30 | 349° F. (176.1° C.) | 1098° F. (592.2° C.) |
| 35 | 364° F. (1844° C.) | 1165° F. (6294° C.) |
| 40 | 393° F. (200.6° C.) | 1208° F. (653.3° C.) |
| 45 | 419° F. (215° C.) | 1247° F. (675° C.) |
| 50 | 427° F. (219.4° C.) | 1280° F. (693.3° C.) |
| 55 | 432° F. (222.2° C.) | 1302° F. (7056° C.) |
| 60 | 441° F. (227.2° C.) | 1319° F. (715° C.) |
| 65 | 453° F. (233.9° C.) | 1330° F. (721.1° C.) |
| 70 | 479° F. (248.3° C.) | 1338° F. (725.6° C.) |
| 75 | 505° F. (262.8° C.) | 1346° F. (730° C.) |
| 80 | 510° F. (265.6° C.) | 1356° F. (735.6° C.) |
| 85 | 513° F. (267.2° C.) | 1367° F. (741.7° C.) |
| 90 | 524° F. (273.3° C.) | 1380° F. (748.9° C.) |
| 95 | 555° F. (290.6° C.) | 1394° F. (7567° C.) |
| 99 | 603° F. (317.2° C.) | 1408° F. (764.4° C.) |
| 99.5 (FBP) | 634° F. (334.4° C.) | 1410° F. (765.6° C.) |

The distillate fraction had a derived cetane number (DCN) of 39.2. The DCN was determined by ASTM D6890-16.

The isoalkane alkylate base oil fraction had the properties shown in Table 2.

TABLE 2

| | |
|---|---|
| Viscosity Index | 92 |
| Kinematic Viscosity at 100° C., mm²/s | 119.0 |
| Kinematic Viscosity at 40° C., mm²/s | 4519 |
| Pour Point, ° C. | −2 |
| Cloud Point, ° C. | <−60 |
| Bromine Index, mg Br/100 g estimated | <100 |
| from NMR | |

The proton NMR of the isoalkane alkylate base oil confirmed that the isoalkane alkylate base oil was completely saturated and contained no vinylic protons. The proton NMR confirmed that the isoalkane alkylate base oil was an alkylate.

It is notable that in the examples described above, no hydroisomerization dewaxing was used to make the isoalkane alkylate base oil or distillate. The isoalkane alkylate base oil was also made without any subsequent hydrogenation after the alkylating, yet still had low a low bromine index. The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed. Unless otherwise specified, all percentages are in weight percent.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

The invention illustratively disclosed herein suitably may be practiced in the absence of any element which is not specifically disclosed herein.

It is claimed:

1. A process to make an isoalkane alkylate base oil, comprising:
a. oligomerizing an olefin feed having a carbon number from 3 to 6 using a metallocene catalyst to make an unsaturated olefin oligomer; and
b. alkylating an isoalkane feed with the unsaturated olefin oligomer in the presence of an acidic alkylation catalyst, and without any addition of hydrogen, to make an alkylate product comprising the isoalkane alkylate base oil having a kinematic viscosity at 100° C. greater than 10 mm²/s, a VI higher than 80, and a bromine index less than 1000 mg Br/100 g.

2. The process of claim 1, additionally comprising distilling the alkylate product and collecting the isoalkane alkylate base oil and a middle distillate.

3. The process of claim 1, wherein the isoalkane feed comprises isobutane, isopentane, hydrocracker naphtha, or mixtures thereof.

4. The process of claim 1, wherein the olefin feed comprises a propylene, a butene, or a mixture thereof.

5. The process of claim 1, wherein the metallocene catalyst comprises a metallocene complex containing a transition metal selected from the group consisting of titanium (Ti), zirconium (Zr), hafnium, (Hf), and combinations thereof.

6. The process of claim 1, wherein the metallocene catalyst comprises a substituted cyclopentadienyl ligand.

7. The process of claim 1, wherein the metallocene catalyst comprises an aluminoxane activator.

8. The process of claim 1, wherein the metallocene catalyst comprises bis(t-butylcyclopentadienyl)zirconium dichloride and MAO.

9. The process of claim 1, wherein the oligomerizing is performed under a hydrogen pressure from 200 to 5000 kPa.

10. The process of claim 1, wherein the olefin feed comprises an inert hydrocarbon solvent.

11. The process of claim 1, wherein the acidic alkylation catalyst is selected from the group consisting of an acidic ionic liquid, a sulfuric acid, a hydrofluoric acid, a triflic acid, another Brønsted acid with a Hammet acidity function less than −10 (H₀<−10), an acidic zeolite, a sulfated zirconia, and a tungstated zirconia.

12. The process of claim 1, wherein the acidic alkylation catalyst comprises an ionic liquid and a Brønsted acid.

13. The process of claim 12, wherein the ionic liquid is a chloroaluminate and the Brønsted acid is hydrogen chloride.

14. The process of claim 1, additionally comprising recycling an excess of the isoalkane feed to the alkylating.

15. The process of claim 1, wherein no hydroisomerization dewaxing is used.

16. The process of claim 1, wherein the isoalkane alkylate base oil comprises from zero to less than 5 wt % linear alkanes.

17. The process of claim 1, wherein the isoalkane alkylate base oil comprises from zero to less than 100 wppm aromatics.

18. The process of claim 2, wherein the middle distillate has a DCN from 30 to 50.

19. The process of claim 1, wherein the isoalkane alkylate base oil comprises a mixture of isoalkanes.

20. The process of claim 1, wherein the isoalkane alkylate base oil comprises localized branching introduced partially from the isoalkane feed and partially from the alkylating.

21. The process of claim 1, wherein the isoalkane alkylate base oil has a pour point less than or equal to 0° C.

22. The process of claim 1, wherein the isoalkane alkylate base oil has a cloud point less than −25° C.

23. The process of claim 1, additionally comprising blending the isoalkane alkylate base oil with at least one additive to make a finished lubricant.

24. An isoalkane alkylate base oil, made by the process of claim 1.
